(19) United States
(12) Patent Application Publication
(10) Pub. No.: US 2021/0357021 A1
(43) Pub. Date: Nov. 18, 2021

(54) PORTABLE AUGMENTED REALITY SYSTEM FOR STEPPING TASK THERAPY

(71) Applicants: Northwestern University, Evanston, IL (US); United States Government as represented by the Department of Veterans Affairs, Washington, DC (US)

(72) Inventors: Matthew Justin Major, Chicago, IL (US); Stefania Fatone, Chicago, IL (US)

(21) Appl. No.: 17/318,520

(22) Filed: May 12, 2021

Related U.S. Application Data
(60) Provisional application No. 63/024,220, filed on May 13, 2020.

(51) Int. Cl. G06F 3/01; G06T 19/00; A63B 71/06; G16H 20/30; G16H 40/67

(52) U.S. Cl. CPC G06F 3/011; G06T 19/006; A63B 71/0622; A63B 2071/0638; G16H 40/67; A63B 2071/0666; G16H 20/30

(57) ABSTRACT

A system to perform neurorehabilitation includes a display that is visible to a user. The system also includes a camera operatively coupled to the display and configured to capture a walking surface upon which the user is walking. The system further includes a processor operatively coupled to the display and to the camera. The processor is configured to project a virtual object onto the walking surface such that the virtual object is visible to the user on the display. The processor is also configured to monitor user movement relative to the virtual object.

### CROSS-REFERENCE TO RELATED APPLICATION

The present application claims the priority benefit of U.S. Provisional Patent App. No. 63/024,220 filed on May 13, 2020, the entire disclosure of which is incorporated by reference herein.

### BACKGROUND

Neurorehabilitation refers to a physician-supervised therapy program that is designed to treat individuals with various diseases or injuries to the nervous system. Treatment approaches based on motor learning theory are currently the prevailing choice in neurorehabilitation and have been shown to improve impairment, function, and quality of life in the setting of chronic neurological disease. Critical motor learning principles include high repetition of functionally relevant movement performed as close to normal as possible, and with feedback on performance. Such activity-based interventions are intended to maximize rehabilitation outcomes and enhance adaptive neural plasticity.

### SUMMARY

An illustrative system to perform neurorehabilitation includes a display that is visible to a user. The system also includes a camera operatively coupled to the display and configured to capture a walking surface upon which the user is walking. The system further includes a processor operatively coupled to the display and to the camera. The processor is configured to project a virtual object onto the walking surface such that the virtual object is visible to the user on the display. The processor is also configured to monitor user movement relative to the virtual object.

An illustrative method of performing neurorehabilitation includes capturing, by a camera, a walking surface upon which a user is walking. The method also includes displaying, on a display operatively coupled to the camera, the walking surface upon which the user is walking. The method also includes projecting, by a processor operatively coupled to the display and to the camera, a virtual object onto the walking surface such that the virtual object is visible to the user on the display. The method further includes monitoring, by the processor, user movement relative to the virtual object.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 depicts an augmented reality display from a user's point-of-view that includes obstacles (e.g., puddles) and targets (e.g., bullseye) overlaid on a real world room with points (top right) as performance feedback in accordance with an illustrative embodiment.

FIG. 2 is a flow diagram depicting operations performed by the system in accordance with an illustrative embodiment.

FIG. 3 is a block diagram of the proposed system in accordance with an illustrative embodiment.

### DETAILED DESCRIPTION

Persons with pathologies that result in balance disorders (e.g. chronic stroke, Parkinson's disease, Huntington's disease, Multiple Sclerosis, lower limb loss, injury, etc.) can benefit from personalized gait therapy for improving balance to help minimize falls and fall-related injuries. However, intervention delivery is often limited due to the need for dedicated space with specialized resources and equipment, and conventional therapy is not engaging or motivational. The current healthcare environment does not support implementation of time-intensive yet critical motor learning principles or activity-based interventions in the traditional rehabilitation clinic. Rather, traditional treatment duration is typically short, with insufficient repetition of movement tasks. The result is that the patient has limited access to skilled care. Consequently, therapists experience low levels of patient compliance, which hinders long-term rehabilitation outcomes. There is thus a need to ensure high dose, high quality practice of adequately challenging patient-driven functional movement for use in neurorehabilitation and other therapies.

Integrating virtual and augmented reality tasks into rehabilitation interventions can improve patient compliance while remaining effective, especially as it offers an option for tele-rehabilitation since the technology can be made portable. Described herein is portable technology that operates through custom software and hardware integrated with common smart phones or other user devices to deliver augmented reality gait therapy for neurorehabilitation of persons with balance disorders in a variety of settings (home, community, inpatient, hospital, etc.). The stepping task intervention is aimed at improving movement control, and is also gamified to boost patient compliance. The technology can be personalized automatically, or by a therapist/physician/clinician in terms of challenge and dosage according to a user's functional capacity as it changes across the rehabilitation journey. While the embodiments described herein relate to stepping tasks, it is to be understood that other tasks/activities may be performed using the methods and systems described herein.

In some embodiments, the proposed methods and systems can be used to supplement in-clinic training with at home practice. Alternatively, the methods and systems can be used primarily or entirely at home by the patient, with remote monitoring, training, etc. by a physician, therapist, or other clinician. Portable devices that are used in both clinical and home settings have the potential to allow for critical repetition of quality practice to occur. Additionally, the therapeutic benefit from a portable device for in-home use can be increased and enhanced through encouragement and reinforcement of task practice, and the ability to gradually progress the training of the user.

The proposed portable technology is capable of delivering augmented reality-based gait therapy and other therapy for neurorehabilitation of persons with impairments in postural control during walking (e.g. chronic stroke, Parkinson's Disease, Huntington's disease, Multiple Sclerosis, lower-limb loss, injury, etc.) in a variety of settings (home, community, inpatient, hospital). Additionally, as discussed, the proposed system is gamified to boost patient compliance. Importantly, this technology can be personalized in terms of challenge and dosage according to the functional capacity of the user as he/she changes across the rehabilitation journey.

The proposed system transforms any environment in which the user is located into a stepping task game for delivery of a gait rehabilitation intervention that is aimed at enhancing balance through training movement control and limb positioning. The system makes use of augmented reality delivered through a smart phone or dedicated virtual reality headset to project virtual objects onto the walking surface that a user must either target or avoid to gain game points. The system also monitors the feet of the user to estimate foot placement in real-time to identify if the user has been successful in stepping onto targets and avoiding obstacles. Specifically, a tracking system that includes one or more cameras can perform image processing to conduct object tracking of the user's foot placement to assess success in completing the stepping task (targeting or avoiding projected virtual objects). Alternatively or in addition, the tracking system can include one or more sensors and/or markers mounted to the user, which are used to track movements of the user. Game points can be displayed in real-time as a score to provide user feedback and encouragement.

More specifically, the proposed system overlays stationary or dynamic virtual objects (e.g., targets or obstacles) onto the physical ground that match the optic flow of walking at any given speed. Stationary virtual objects can be a fixed position in an environment that moves as the user moves. Alternatively, dynamic virtual objects can be moving even if the user is stationary. The speed is controllable, and can be set by the user or physician. Alternatively, the system may automatically detect a walking speed (gait) and display virtual objects at a rate controlled based on the detected walking speed. The virtual objects placed in the walking path of the user create a game for stepping tasks that can be personalized by changing the challenge level. The tracking of foot placement provides a measurement of accuracy of either hitting the targets or avoiding obstacles. Custom software to implement the system can be integrated into a smart phone or dedicated virtual reality headset. In one implementation, commonly used smart phones can be inserted into off-the-shelf goggle headsets to implement the system.

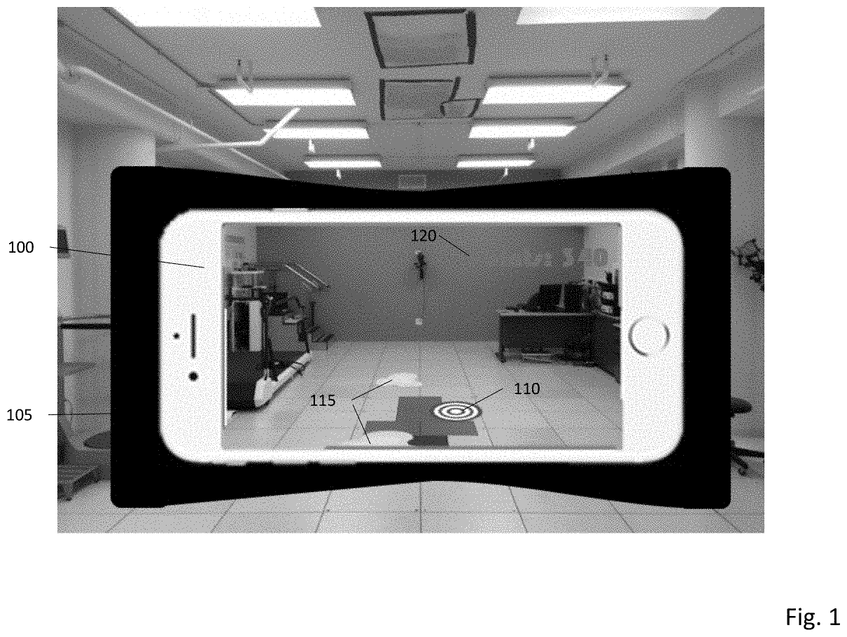

An example stepping task is projection of puddles and bullseyes overlaid on the ground to denote obstacles and targets, respectively. FIG. 1 depicts an augmented reality display from a user's point-of-view that includes obstacles (e.g., puddles) and targets (e.g., bullseye) overlaid on a real world room with points (top right) as performance feedback in accordance with an illustrative embodiment. Alternatively, objects other than puddles and bullseyes can be used such as stars, circles, animated characters, lines, virtual pathways or walkways, etc. As shown, the system includes a smartphone 100 mounted in a set of goggles 105. The smartphone 100 can be any type of portable phone with a camera that allows the user to view his/her environment while looking at the screen of the phone. The goggles 105 include a mount to hold the smartphone 100 in place and one or more straps to secure the system to the head of the user such that the mounted smartphone 100 is in front of the eyes of the user. In some embodiments, the goggles 105 may include built-in speakers to deliver audio to the user, such as metronome tones to set cadence, alert tones as feedback when an obstacle is missed/hit or a target is hit/missed, or music warped according to a tracked walking dynamic. Alternatively, the speaker(s) of the smartphone 100 may be used to deliver the audio, when used. In an alternative embodiment, instead of a smartphone 100 and goggles 105, the system may be implemented as a dedicated augmented reality or virtual reality headset.

As shown, the user is able to view his/her surroundings through the smartphone 100. In addition to the actual environment, the system overlays targets 110 that the user is asked to step on with one or both feet, and obstacles 115 that the user is asked to avoid. In an illustrative embodiment, when approached, the virtual objects do not have any height, but are flush with the walking surface in the environment where the user is located. The rate at which the virtual objects appear can be set by the user or remotely controlled by a physician. In one embodiment, the rate at which virtual objects appear can be automatically determined and controlled by the system as it detects the natural walking pace of the user. As the user progresses through a program, the user is rewarded for successful steps (i.e., steps that hit a target or avoid an obstacle), and a points display 120 provides the user with a score that the user can view in real time to track his/her progress.

Object tracking via real-time image processing is used to identify the feet of users (either the feet/shoes themselves or unique markers attached to the feet/shoes) to estimate position of the feet relative to the overlaid virtual objects. The system can also distinguish between the left and right foot of the user based on sensor data, foot shape, foot orientation, foot location, etc. When the foot is detected to be within/outside the boundary of the virtual target/obstacle, that event is registered as a successful target hit or obstacle avoidance, respectively. This therapy is personalized by modifying the challenge required to accomplish the step task through adjustment of various features such as the width of obstacles that effectively controls step width, and the distance between objects, which effectively controls step length. In one embodiment, accuracy of limb position for a given walking trial is calculated as the number of successful hits and avoidances divided by the total number of objects navigated. Successful hits and avoidances generate point totals that are displayed to the user as performance feedback as shown in FIG. 1, thereby motivating users to engage in the stepping task therapy.

The proposed system is not limited to the embodiment depicted in FIG. 1. For example, the system may implement another task in the form of projected rails at a fixed distance apart that the user is asked to stay in between. Another task may involve a projected single straight line that the user is asked to follow and step upon. Another task may involve a projected circular line that the user is asked to follow and step upon. Another task may include projecting a checkerboard pattern and asking the user to only step in certain squares of the pattern. Yet another task may include a series of projected interconnected lines that the user is asked to follow and step upon. These are intended as examples, and it is to be understood that the methods and systems described herein may be used for other neurorehabilitation tasks in addition to those explicitly mentioned.

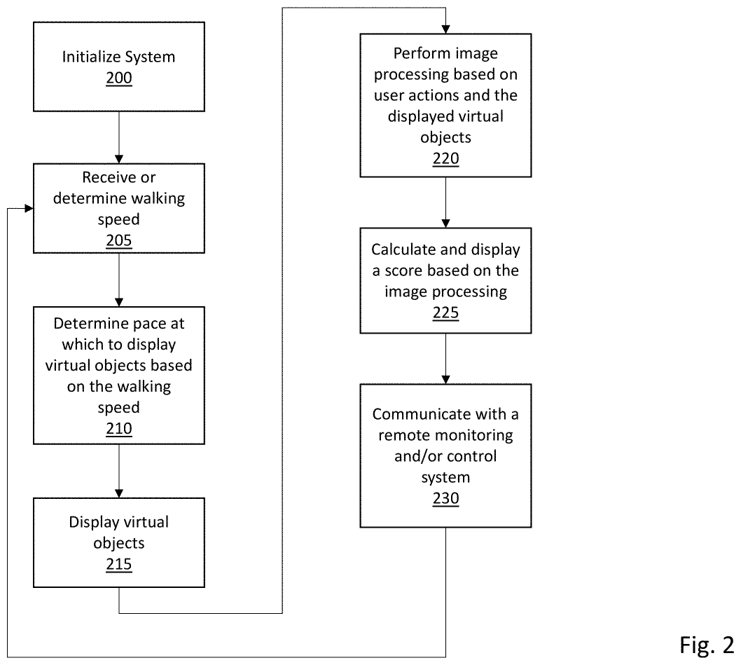

FIG. 2 is a flow diagram depicting operations performed by the system in accordance with an illustrative embodiment. In alternative embodiments, fewer, additional, and/or different operations may be performed. Also, the use of a flow diagram is not meant to be limiting with respect to the order of operations performed. In an operation 200, the system is initialized based on received inputs and instructions. System initialization can be performed locally by a user or physician, or remotely by a physician who is in remote communication with the system. System initialization can include mounting a smartphone into a set of goggles, receiving commands to turn on the goggles and/or smartphone, receiving a command to start a dedicated therapy application on the smartphone, receiving a selection of a type of therapy task within the application that is to be performed by the user, etc. In an embodiment where the system is implemented as dedicated augmented reality goggles, the system initialization can involve receiving a command to turn the device on and receiving a selection of the specific type of therapy task that is to be performed out of a plurality of different available tasks. In an embodiment in which sensors are used to help track user movement, the initialization can also include mounting of the sensors on the user and/or detection of the sensors by the smartphone or other processing component of the system.

In an operation 205, the system receives or determines a walking (or running) speed of the user. In some embodiments, the speed is set by the user or by the physician based on a therapy goal. In an embodiment in which the physician is remote, the speed setting can be received from a remote location at which the physician is located. In an alternative embodiment, the system can be used during natural walking (or running) of the user, and the system can automatically determine the user speed based on the actions of the user. In such an embodiment, the speed of the user can be determined using any image processing techniques known in the art.

In an operation 210, the system determines the pace at which to display virtual objects based on the walking speed. In an illustrative embodiment, the system is designed to display virtual objects at a pace that matches the desired (or actual) walking speed of the user. In another illustrative embodiment, the pace of display is dynamic and can change as the walking speed of the user changes. In an embodiment in which the virtual object(s) are continuous (e.g., a straight or curved line that the user is asked to follow or avoid), the system may just display the object(s) without taking into consideration the walking speed of the user.

In an operation 215, the system displays the virtual object(s). As discussed, the virtual object(s) can be displayed as flat representations on the surface upon which the user is walking. In embodiments in which a plurality of virtual objects are being projected, the projections can be displayed at the pace determined in the operation 210. Additionally, the system can control the size (e.g., width or length) of the virtual objects and/or the distance between virtual objects to work on specific aspects of the user's movements. The size of virtual objects and/or the distance in between virtual objects can be statically set based upon the specific task selected by (or for) the user, or they can be controlled dynamically by the user or the physician during performance of the task.

In an operation 220, the system performs image processing based on user actions and the displayed virtual objects. In an illustrative embodiment, the user actions are steps taken. In alternative embodiments, different actions may be monitored such as arm or hand movements, head movements, hip movements, etc. In some embodiments, the user has one or more sensors attached to his/her feet (or other body part) and the image processing is based at least in part on the locations of the sensors captured in images by the system camera or information transmitted by the sensors. The sensors can be markers that are readily detected by the camera, transmitters that detect a position and transmit it to the processor of the system (e.g., the smartphone), etc. In an alternative embodiment, the system can be trained to recognize the feet of the user (e.g., through shape recognition) or other body part without the use of sensors, which results in a system that is easier for the user to use.

In an illustrative embodiment, the system uses image processing to obtain first coordinates (or other location identifying data) corresponding to an area at which a virtual object is positioned and second coordinates corresponding to one or more areas at which the user's feet or other body parts are located. The system compares these two sets of coordinates to determine whether the user is successful in hitting or avoiding the projected virtual objects. For example, if the user is supposed to hit a target, the system can determine whether the coordinates of at least one of the user's feet is entirely within the coordinates of the projected virtual object to gauge success. Similarly, if the user is supposed to avoid an object, the system can determine whether the coordinates of the user's feet entirely avoid the object to gauge success. In some instances, the coordinates of the user's feet may only partially overlap (or partially avoid) the coordinates of the virtual object.

In an operation 225, the system calculates and displays a score for the user based on the image processing. In one implementation, the score can be an absolute value that is either entirely awarded or not awarded at all, depending on how the user performed. For example, the system may issue a score of 100 for each successful movement and a 0 for each unsuccessful movement, where success is defined as complete overlap (or complete avoidance) of the coordinates of the feet of the user with the coordinates of the virtual object. Alternatively, unsuccessful movements may result in negative points. In another alternative embodiment, the system may award points based on an amount of overlap (or an amount of avoidance) of the coordinates of the user's feet and the coordinates of the projected virtual object. For example, if the user is supposed to step on a target with his/her left foot, the system might determine that the coordinates of the user's left foot overlapped with the coordinates of the projected target by 72%, which may result in a score of 72 out of 100. Similarly, if the user is supposed to avoid an object with his/her right foot, the system might determine that the coordinates of the user's right foot overlapped with the projected object by 7%, which may result in a score of 93 out of 100 for that action. These are just examples, and in alternative embodiments different scoring algorithms may be used. The score can be displayed on the view screen (or display) that the user is viewing, as shown in FIG. 1. In an alternative embodiment, the score can be saved in memory, but not displayed in real time for the user.

In an operation 230, the system communicates with a remote monitoring and/or control system. In an illustrative embodiment, the remote system is located at a clinic or other physician's office and allows the physician to remote monitor and/or control the system. The remote system can be a desktop computer, laptop computer, tablet, smartphone, etc. The remote system can be used to initialize the task system for the user, to set the walking speed for the user, to set the specific task that the user is to perform, to control the projection rate of the virtual objects, to control the size of the virtual objects, to control the distance between virtual objects, etc. The remote system can also receive real time data corresponding to the user's performance, such as the amount of overlap of the user's feet with the projected objects, the user's score, the user's actual walking speed, etc. As shown, in an illustrative embodiment, the process performed by the system is iterative and continuous such that the system can continuously monitor for the walking speed (or receive a revised walking speed input) and adjust the pace at which objects are displayed accordingly. Similarly, the image processing, score calculation, and remote communication can be continuously performed until the user (or physician) ends the program session.

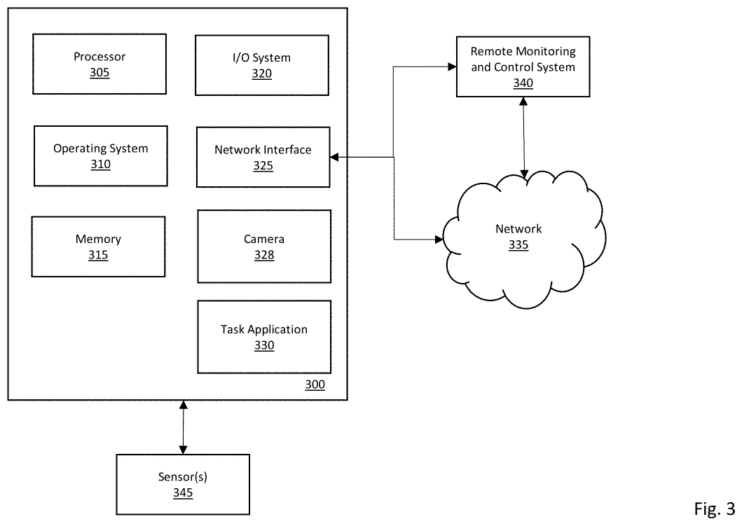

FIG. 3 is a block diagram of the proposed system in accordance with an illustrative embodiment. FIG. 3 depicts a user computing device 300 in communication with a network 335 and a remote monitoring and control system 340. The remote monitoring and control system 340 can be any type of computing device, and can include a processor, memory, transceiver, user interface, etc. As discussed, the remote monitoring and control system 340 can be used by a physician to remotely monitor and control the user computing device 300. The user computing device 300 is in local communication with one or more sensors 345, and includes a processor 305, an operating system 310, a memory 315, an input/output (I/O) system 320, a network interface 325, a camera 328, and a task application 330. In alternative embodiments, the user computing device 300 may include fewer, additional, and/or different components.

The components of the user computing device 300 communicate with one another via one or more buses or any other interconnect system. The user computing device 300 can be any type of networked computing device, a convenient version of which is a smartphone mounted in a set of goggles. In an alternative embodiment, instead of a smartphone, the user computing device 300 can be a tablet, a music player, a portable gaming device, a dedicated device specific to the task application, etc. In another alternative embodiment, the user computing device 300 can be a dedicated set of goggles (e.g., a virtual reality system) that perform the functions described herein.

The processor 305 can be in electrical communication with and used to control any of the system components described herein. The processor 305 can be any type of computer processor known in the art, and can include a plurality of processors and/or a plurality of processing cores. The processor 305 can include a controller, a microcontroller, an audio processor, a graphics processing unit, a hardware accelerator, a digital signal processor, etc. Additionally, the processor 305 may be implemented as a complex instruction set computer processor, a reduced instruction set computer processor, an x86 instruction set computer processor, etc. The processor 305 is used to run the operating system 310, which can be any type of operating system.

The operating system 310 is stored in the memory 315, which is also used to store programs, user data, network and communications data, peripheral component data, the task application 330, and other operating instructions. The memory 315 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc. In some embodiments, at least a portion of the memory 315 can be in the cloud to provide cloud storage for the system. Similarly, in one embodiment, any of the computing components described herein (e.g., the processor 305, etc.) can be implemented in the cloud such that the system can be run and controlled through cloud computing.

The I/O system 320 is the framework which enables users and peripheral devices to interact with the user computing device 300. The I/O system 320 can include one or more displays (e.g., light-emitting diode display, liquid crystal display, touch screen display, etc.) that allow the user to view his/her environment while performing the tasks, a speaker, a microphone, etc. that allow the user to interact with and control the user computing device 300. The I/O system 320 also includes circuitry and a bus structure to interface with peripheral computing devices such as power sources, USB devices, data acquisition cards, peripheral component interconnect express (PCIe) devices, serial advanced technology attachment (SATA) devices, high definition multimedia interface (HDMI) devices, proprietary connection devices, etc.

The network interface 325 includes transceiver circuitry (e.g., a transmitter and a receiver) that allows the computing device to transmit and receive data to/from other devices such as the remote monitoring and control system 340, the sensor(s) 345, other remote computing systems, servers, websites, etc. The data transmitted to the remote monitoring and control system 340 can include detected speed data, detected coordinate data (of the user and/or the virtual objects), user score, video of the user performing the task, audio from the user, sensor data, etc. The data received from the remote monitoring and control system 340 can include indication of a type of task to be performed by the user, a walking speed for the user to achieve, a rate at which to display virtual objects, a size of the virtual objects, a type of virtual object, a distance between projected virtual objects, etc. The network interface 325 enables communication through the network 335, which can be one or more communication networks. The network 335 can include a cable network, a fiber network, a cellular network, a wi-fi network, a landline telephone network, a microwave network, a satellite network, etc. The network interface 325 also includes circuitry to allow device-to-device communication such as Bluetooth® communication.

The camera 328 is used in conjunction with the display of the user computing device 300 to provide the user with a view of their surroundings and to capture imagery of the user and/or the sensor(s) 345 as they complete tasks. Any type of camera may be used. In an illustrative embodiment, the camera is used in conjunction with the sensor(s) to monitor user movement. The sensor(s) 345 can be passive sensors that act as markers which are readily detected by the camera 328 based on the light emitting/reflecting characteristics of the markers. Alternatively, the sensor(s) 345 can be active sensors that transmit detected location data to the user computing device 300, such as coordinate information, speed information, etc. The transmissions can occur through Bluetooth® communication, other short range communication techniques, other network communication algorithms, etc. The sensor(s) 345 can also be used to distinguish between the left foot and the right foot (or other body parts) of the user. In an alternative embodiment, the sensor(s) may not be used, and the camera 328 can be trained to identify the feet of the user. The camera 328 can also be trained to distinguish between the left foot and the right foot of the user based on shape, position, etc.

The task application 330 can include software and algorithms in the form of computer-readable instructions which, upon execution by the processor 305, performs any of the various operations described herein such as initializing the system, determining walking speed, displaying virtual objects, processing received selections and inputs from the user, processing captured image data, analyzing sensor readings from the sensor(s) 345, calculating and/or displaying a user score, receiving instructions from the remote monitoring and control system 340, sending captured imagery and/or other results to the remote monitoring and control system 340, etc. The task application 330 can utilize the processor 305 and/or the memory 315 as discussed above. In an alternative implementation, the task processing application 330 can be remote or independent from the user computing device 300, but in communication therewith.

As discussed herein, the proposed methods and systems can be used for physical therapy in a clinical environment, for physical therapy in a home environment, for augmented reality gaming for both therapy and entertainment, etc. Conventional therapy relies on equipment located in specialty clinics. Conversely, the proposed methods and systems offer a portable solution that can be implemented in virtually any environment in which the user is located. While current augmented reality stepping task training requires a large treadmill and/or fixed projector, the proposed technology uses a system that involves use of a smart phone and headset or dedicated virtual reality goggles. Additionally, conventional therapies for balance disorders are not engaging for patients, while the proposed technology is gamified to enhance engagement, motivation, and patient compliance.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more”.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A system to perform neurorehabilitation comprising:
a display that is visible to a user;
a camera operatively coupled to the display and configured to capture a walking surface upon which the user is walking; and
a processor operatively coupled to the display and to the camera, wherein the processor is configured to:
project a virtual object onto the walking surface such that the virtual object is visible to the user on the display; and
monitor user movement relative to the virtual object.

2. The system of claim 1, further comprising a transceiver that communicates with a remote system, wherein the transceiver is configured to receive a selection of a type of task from the remote system, and wherein the selected type of task is to be performed by the user.

3. The system of claim 1, further comprising a transceiver that communicates with a remote system, wherein the transceiver is configured to transmit video of the user movement to the remote system.

4. The system of claim 1, further comprising goggles that mount to a head of the user and that incorporate the display, the camera, and the processor such that the display is in a position that is visible to the user.

5. The system of claim 1, wherein the processor is configured to receive a speed of the user, and to determine a rate at which to project virtual objects based at least in part on the speed of the user.

6. The system of claim 5, wherein the processor receives the speed of the user as an input from the user.

7. The system of claim 5, wherein the processor receives the speed of the user as an input from a remote computing system.

8. The system of claim 5, wherein the processor calculates the speed of the user based on an analysis of detected user movements.

9. The system of claim 5, wherein the processor is configured to cause projection of a plurality of virtual objects in succession at the determined rate.

10. The system of claim 1, wherein the processor determines a first area of a first location at which the virtual object is projected and a second area of a second location where the user places a foot.

11. The system of claim 10, wherein the processor determines whether the first area overlaps with the second area to monitor the user movement.

12. The system of claim 10, wherein the processor determines an amount of overlap between the first area and the second area to monitor the user movement.

13. The system of claim 10, wherein the processor is configured to compare the first area to the second area, and to generate a score for the user based on the comparison.

14. The system of claim 1, further comprising one or more sensors mounted on the user, wherein the camera is configured to identify the one or more sensors, and wherein the processor uses the one or more identified sensors to monitor the movement of the user.

15. The system of claim 1, further comprising one or more sensors mounted on the user, wherein the processor is configured to process information transmitted by the one or more sensors to monitor the movement of the user.

16. A method of performing neurorehabilitation, the method comprising:
capturing, by a camera, a walking surface upon which a user is walking;
displaying, on a display operatively coupled to the camera, the walking surface upon which the user is walking;
projecting, by a processor operatively coupled to the display and to the camera, a virtual object onto the walking surface such that the virtual object is visible to the user on the display; and
monitoring, by the processor, user movement relative to the virtual object.

17. The method of claim 16, further comprising:
determining, by the processor, a speed of the user; and
determining, by the processor, a rate at which to project virtual objects based at least in part on the determined speed of the user.

18. The method of claim 17, further comprising causing, by the processor, projection of a plurality of virtual objects in succession on the display at the determined rate.

19. The method of claim 16, further comprising determining, by the processor, a first area of a first location at which the virtual object is projected and a second area of a second location where the user places a foot.

20. The method of claim 19, further comprising comparing, by the processor, the first area to the second area, and generating a score for the user based on the comparison.
